(19) United States
(12) Patent Application Publication
(10) Pub. No.: US 2019/0194732 A1
(43) Pub. Date: Jun. 27, 2019

(54) DEVICE AND METHOD FOR NUCLEIC ACID MANIPULATION

(71) Applicant: Ginkgo Bioworks, Inc., Boston, MA (US)

(72) Inventor: Joseph Jacobson, Newton, MA (US)

(21) Appl. No.: 16/321,150

(22) PCT Filed: Jul. 28, 2017
(86) PCT No.: PCT/US17/44334

§ 371 Date: Jan. 28, 2019

Related U.S. Application Data
(60) Provisional application No. 62/367,715, filed on Jul. 28, 2016.

(51) Int. Cl. C12Q 1/6834; C07H 21/04; B01J 19/00; C07H 21/02

(52) U.S. Cl. CPC C12Q 1/6834; C07H 21/04; B01J 19/00; C07H 21/02; C12Q 2537/143; C12Q 2563/107; B01J 2219/00722; B01J 2219/005; B01J 2219/00545; B01J 2219/00547; B01J 2219/00576; B01J 2219/00585; C12Q 2563/149

(57) ABSTRACT

Devices and methods are provided for multiplex nucleic acid assembly. Specifically, the device includes a plurality of differentially labeled particles or beads, such that when in use, the labeled particles can barcode different oligonucleotides. The device can be used for nucleic acid singulation during and/or after assembly.

### CROSS REFERENCE TO RELATED APPLICATIONS

This application claims priority to and the benefit of U.S. Provisional Application No. 62/367,715, the disclosure of which is incorporated herein by reference in its entirety.

### FIELD

The devices and methods disclosed herein relate to nucleic acid manipulation, particularly during multiplex nucleic acid assembly.

### BACKGROUND

Recombinant and synthetic nucleic acids have many applications in research, industry, agriculture, and medicine. Recombinant and synthetic nucleic acids can be used to express and obtain large amounts of polypeptides, including enzymes, antibodies, growth factors, receptors, and other polypeptides that may be used for a variety of medical, industrial, or agricultural purposes. Recombinant and synthetic nucleic acids also can be used to produce genetically modified organisms including modified bacteria, yeast, mammals, plants, and other organisms. Genetically modified organisms may be used in research (e.g., as animal models of disease, as tools for understanding biological processes, etc.), in industry (e.g., as host organisms for protein expression, as bioreactors for generating industrial products, as tools for environmental remediation, for isolating or modifying natural compounds with industrial applications, etc.), in agriculture (e.g., modified crops with increased yield or increased resistance to disease or environmental stress, etc.), and for other applications. Recombinant and synthetic nucleic acids also may be used as therapeutic compositions (e.g., for modifying gene expression, for gene therapy, etc.) or as diagnostic tools (e.g., as probes for disease conditions, etc.).

Indeed, nucleic acid synthesis is an important area of synthetic biology. According to the U.S. Department of Energy (DOE) in its Report to Congress on dated July 2013, “synthetic biology” is “the design and wholesale construction of new biological parts and systems, and the re-design of existing, natural biological systems for tailored purposes, integrates engineering and computer-assisted design approaches with biological research.” DNA synthesis and assembly have been identified as a fundamental challenge for the continued development of synthetic biology in the DOE report. Specifically, “[o]ne of the major limitations to experimentation in synthetic biology is the synthesis and assembly of large DNA constructs, which remains expensive, slow and error prone. Engineering new bio-production systems would require new approaches for synthesizing and assembling genetic designs rapidly, cheaply, and accurately.”

Numerous techniques have been developed for modifying existing nucleic acids (e.g., naturally occurring nucleic acids) to generate recombinant nucleic acids. For example, combinations of nucleic acid amplification, mutagenesis, nuclease digestion, ligation, cloning and other techniques may be used to produce many different recombinant nucleic acids. Chemically synthesized polynucleotides are often used as primers or adaptors for nucleic acid amplification, mutagenesis, and cloning.

Techniques also are being developed for de novo nucleic acid synthesis on solid supports. For example, single-stranded oligonucleotides of predetermined nucleic acid sequences can be synthesized in situ on a common support wherein each predetermined nucleic acid sequence is synthesized on a separate or discrete feature (or spot) on the support.

Techniques are also available for de novo nucleic acid assembly whereby nucleic acids are made (e.g., chemically synthesized on a support) and assembled to produce longer target nucleic acids of interest. For example, different multiplex assembly techniques are being developed for assembling oligonucleotides into larger synthetic nucleic acids that can be used in research, industry, agriculture, and/or medicine. However, one limitation of currently available support-based synthesis and assembly techniques is the ability to identify and select one or more targets of interest. As such, high precision, high selectivity nucleic acid singulation and assembly techniques are needed.

### SUMMARY

In one aspect, a device is provided for multiplex nucleic acid assembly. The device can include:
a plurality of differentially labeled particles comprising a first, a second and a third particle, having a first, a second and a third anchor oligonucleotide immobilized thereon, respectively;
a plurality of first construction oligonucleotides designed to at least partially anneal to one another in a first predetermined order, and to at least partially anneal to the first anchor oligonucleotide at a first terminal oligonucleotide, thereby forming a first nucleic acid on the first particle;
a plurality of second construction oligonucleotides designed to at least partially anneal to one another in a second predetermined order, and to at least partially anneal to the second anchor oligonucleotide at a second terminal oligonucleotide, thereby forming a second nucleic acid on the second particle; and
a plurality of third construction oligonucleotides designed to at least partially anneal to one another in a third predetermined order, and to at least partially anneal to the third anchor oligonucleotide at a third terminal oligonucleotide, thereby forming a third nucleic acid on the third particle.

Another aspect relates to a method for multiplex nucleic acid assembly, comprising:
providing a plurality of differentially labeled particles comprising a first particle, a second particle and a third particle, having a first anchor oligonucleotide, a second anchor oligonucleotide and a third anchor oligonucleotide immobilized thereon, respectively;
providing a plurality of first construction oligonucleotides designed to at least partially anneal to one another in a first predetermined order, and to at least partially anneal to the first anchor oligonucleotide at a first terminal oligonucleotide, thereby forming a first nucleic acid on the first particle;
providing a plurality of second construction oligonucleotides designed to at least partially anneal to one another in a second predetermined order, and to at least partially anneal to the second anchor oligonucleotide at a second terminal oligonucleotide, thereby forming a second nucleic acid on the second particle; and
providing a plurality of third construction oligonucleotides designed to at least partially anneal to one another in a third predetermined order, and to at least partially anneal to the third anchor oligonucleotide at a third terminal oligonucleotide, thereby forming a third nucleic acid on the third particle.

In various embodiments, the particles can each have a different fluorophore attached thereto. In certain embodiments, the particles may have a different mixture of two fluorophores attached thereto. The pluralities of differentially labeled particles can be provided in a single reaction volume. In some embodiments, the first, second and third oligonucleotides can be designed to have uniquely complementary sequences. In certain embodiments, each particle can be provided in a separate reaction volume. In some embodiments, each separate reaction volume can be provided in a different well.

### BRIEF DESCRIPTION OF THE FIGURES

FIGS. 1A and 1B illustrate, in one embodiment, the assembly of oligonucleotides immobilized on beads with additional oligonucleotides.

FIG. 2 illustrates an exemplary method of singulation of assembled oligonucleotide.

### DETAILED DESCRIPTION

Devices and methods disclosed herein relate to nucleic acid manipulation, particularly during multiplex nucleic acid assembly. In some embodiments, bead-based singulation can be used to selectively pick one or more targets, before, during and/or after multiplex nucleic acid assembly from, e.g., synthetic oligonucleotides that may have been synthesized and/or immobilized on a bead or other individualized solid supports.

### Definitions

For convenience, certain terms employed in the specification, examples, and appended claims are collected here. Unless defined otherwise, all technical and scientific terms used herein have the same meaning as commonly understood by one of ordinary skill in the art to which this disclosure belongs.

The articles “a” and “an” are used herein to refer to one or to more than one (i.e., at least one) of the grammatical object of the article. By way of example, “an element” means one element or more than one element.

As used herein, the term “about” means within 20%, more preferably within 10% and most preferably within 5%. The term “substantially” means more than 50%, preferably more than 80%, and most preferably more than 90% or 95%.

As used herein, “a plurality of” means more than 1, e.g., 2, 3, 4, 5, 6, 7, 8, 9, 10, 11, 12, 13, 14, 15, 16, 17, 18, 19, 20, or more, e.g., 25, 30, 40, 50, 60, 70, 80, 90, 100, 200, 300, 400, 500, or more, or any integer therebetween.

“Assembly” or “assemble” means a process in which short DNA sequences (construction oligonucleotides) are attached in a particular order to form a longer DNA sequence (target). “Subassembly” or “subassemble” means an intermediate step or product where a subset of the construction oligonucleotides are attached to form a subconstruct that is a portion of the final target.

“CEL” or “cohesive end ligation” refers to the process of joining DNA fragments in a predesigned order using cohesive ends that are at least partially complementary to one another. The cohesive ends can be generated by restriction enzyme digestion or can be directly synthesized, e.g., on a solid support.

As used herein, a “chip” refers to a DNA microarray with many oligonucleotides attached to a planar surface. The oligonucleotides on a chip can be any length. In some embodiments, the oligonucleotides are about 10-1,000, 20-800, 50-500, 100-300 or about 200 nucleotides, or longer or shorter, or any number in between. The oligonucleotides may be single stranded or double stranded.

As used herein, a “complementary” or “complementarity” means that two nucleic acid sequences are capable of at least partially base-pairing with one another according to the standard Watson-Crick complementarity rules. For example, two sticky ends can be partially complementary, wherein a region of one overhang complements and anneals with a region or all of another overhang. The gap(s), if any, can be filled in by chain extension in the presence of a polymerase and single nucleotides, followed by or simultaneously with a ligation reaction.

As used herein, a “construct” refers to a DNA sequence which includes a complete target sequence. Generally it is implied that the construct has been assembled. A “subconstruct” means a portion of the complete target sequence that typically is an intermediate product during hierarchical assembly.

As used herein, a “feature” refers to a discrete location (or spot) on a solid support, e.g., a chip, multiwell tray, or microarray. In some embodiments, oligonucleotides can be synthesized on and/or immobilized to the feature. An arrangement of discrete features can be presented on the solid support for storing, routing, amplifying, releasing and otherwise manipulating oligonucleotides or complementary oligonucleotides for further reactions. In some embodiments, each feature is addressable; that is, each feature is positioned at a particular predetermined, prerecorded location (i.e., an “address”) on the support. Therefore, each oligonucleotide is localized to a known and defined location on the support. The sequence of each oligonucleotide can be determined from its position on the support. The size of the feature can be chosen to allow formation of a microvolume (e.g., 1-1000 microliters, 1-1000 nanoliters, 1-1000 picoliters) droplet on the feature, each droplet being kept separate from each other. As used herein, features are typically, but need not be, separated by interfeature spaces to ensure that droplets between two adjacent features do not merge. Interfeatures will typically not carry any oligonucleotide on their surface and will correspond to inert space. In some embodiments, features and interfeatures may differ in their hydrophilicity or hydrophobicity properties.

As used herein, “nucleic acid,” “nucleic acid sequence,” “oligonucleotide,” “polynucleotide,” “gene” or other grammatical equivalents as used herein means at least two nucleotides, either deoxyribonucleotides or ribonucleotides, or analogs thereof, covalently linked together. Polynucleotides are polymers of any length, including, e.g., 10, 20, 50, 100, 200, 300, 500, 1000, etc. As used herein, an “oligonucleotide” may be a nucleic acid molecule comprising at least two covalently bonded nucleotide residues. In some embodiments, an oligonucleotide may be between 10 and 1,000 nucleotides long. For example, an oligonucleotide may be between 10 and 500 nucleotides long, or between 500 and 1,000 nucleotides long. In some embodiments, an oligonucleotide may be between about 20 and about 800 nucleotides long (e.g., from about 20 to 400, from about 400 to 800 nucleotides long). In some embodiments, an oligonucleotide may be between about 50 and about 500 nucleotides long (e.g., from about 50 to 250, from about 250 to 500 nucleotides long). In some embodiments, an oligonucleotide may be between about 100 and about 300 nucleotides long (e.g., from about 100 to 150, from about 150 to 300 nucleotides long). However, shorter or longer oligonucleotides may be used. An oligonucleotide may be a single-stranded or double-stranded nucleic acid. As used herein the terms “nucleic acid”, “polynucleotide” and “oligonucleotide” are used interchangeably and refer to naturally-occurring or non-naturally occurring, synthetic polymeric forms of nucleotides. In general, the term “nucleic acid” includes both “polynucleotide” and “oligonucleotide” where “polynucleotide” may refer to longer nucleic acid (e.g., more than 1,000 nucleotides, more than 5,000 nucleotides, more than 10,000 nucleotides, etc.) and “oligonucleotide” may refer to shorter nucleic acid (e.g., 10-500 nucleotides, 20-400 nucleotides, 40-200 nucleotides, 50-100 nucleotides, etc.).

The nucleic acid molecules of the present disclosure may be formed from naturally occurring nucleotides, for example forming deoxyribonucleic acid (DNA) or ribonucleic acid (RNA) molecules. Alternatively, naturally-occurring nucleic acids may include structural modifications to alter their properties, such as in peptide nucleic acids (PNA) or in locked nucleic acids (LNA). The solid phase synthesis of nucleic acid molecules with naturally occurring or artificial bases is well known in the art. The terms should be understood to include equivalents, analogs of either RNA or DNA made from nucleotide analogs and as applicable to the embodiment being described, single-stranded or double-stranded polynucleotides. Nucleotides useful in the disclosure include, for example, naturally-occurring nucleotides (for example, ribonucleotides or deoxyribonucleotides), or natural or synthetic modifications of nucleotides, or artificial bases. In some embodiments, the sequence of the nucleic acids does not exist in nature (e.g., a cDNA or complementary DNA sequence, or an artificially designed sequence).

Usually in a nucleic acid nucleosides are linked by phosphodiester bonds. Whenever a nucleic acid is represented by a sequence of letters, it will be understood that the nucleosides are in the 5′ to 3′ order from left to right. In accordance to the IUPAC notation, “A” denotes deoxyadenosine, “C” denotes deoxycytidine, “G” denotes deoxyguanosine, “T” denotes deoxythymidine, “U” denotes the ribonucleoside, uridine. In addition, there are also letters which are used when more than one kind of nucleotide could occur at that position: “W” (i.e. weak bonds) represents A or T, “S” (strong bonds) represents G or C, “M” (for amino) represents A or C, “K” (for keto) represents G or T, “R” (for purine) represents A or G, “Y” (for pyrimidine) represents C or T, “B” represents C, G or T, “D” represents A, G or T, “H” represents A, C or T, “V” represents A, C, or G and “N” represents any base A, C, G or T (U). It is understood that nucleic acid sequences are not limited to the four natural deoxynucleotides but can also comprise ribonucleoside and non-natural nucleotides. A “I” in a nucleotide sequence or nucleotides given in brackets refer to alternative nucleotides, such as alternative U in a RNA sequence instead of T in a DNA sequence. Thus, U/T or U(T) indicate one nucleotide position that can either be U or T. Likewise, A/T refers to nucleotides A or T; G/C refers to nucleotides G or C. Due to the functional identity between U and T any reference to U or T herein shall also be seen as a disclosure as the other one of T or U. For example, the reference to the sequence UUCG (on an RNA) shall also be understood as a disclosure of the sequence TTCG (on a corresponding DNA). For simplicity only, only one of these options is described herein. Complementary nucleotides or bases are those capable of base pairing such as A and T (or U); G and C; G and U.

As used herein, the term “solid support”, “support” and “substrate” are used interchangeably and refers to a porous or non-porous solid (e.g., solvent insoluble) material on which polymers such as nucleic acids are synthesized or immobilized. As used herein “porous” means that the material contains pores having substantially uniform diameters (for example in the nm range). Porous materials can include but are not limited to, paper, synthetic filters and the like. In such porous materials, the reaction may take place within the pores. The support can have any one of a number of shapes, such as pin, strip, plate, disk, rod, bends, cylindrical structure, particle, including bead, nanoparticle and the like. In some embodiments, the support is planar (e.g., a chip). The support can have variable widths. The solid support can be an organized matrix or network of wells, such as a microarray. In some embodiments, the support can include a plurality of beads or particles, optionally positioned in one or more multiwall plates.

The support can be hydrophilic or capable of being rendered hydrophilic. The support can include inorganic powders such as silica, magnesium sulfate, and alumina; natural polymeric materials, particularly cellulosic materials and materials derived from cellulose, such as fiber containing papers, e.g., filter paper, chromatographic paper, etc.; synthetic or modified naturally occurring polymers, such as nitrocellulose, cellulose acetate, poly (vinyl chloride), polyacrylamide, cross linked dextran, agarose, polyacrylate, polyethylene, polypropylene, poly (4-methylbutene), polystyrene, polymethacrylate, poly(ethylene terephthalate), nylon, poly(vinyl butyrate), polyvinylidene difluoride (PVDF) membrane, glass, controlled pore glass, magnetic controlled pore glass, ceramics, metals, and the like; either used by themselves or in conjunction with other materials.

As used herein, “particles” or “beads” refer to any solid or semi-solid particles to which nucleic acid sequences can be attached, which are suitable for a multiplex assay and which are stable and insoluble under hybridization, detection, and/or gating conditions. The particles or beads can be of any shape, such as cylindrical, spherical, and so forth, size, composition, or chemical characteristics. The particle size or composition can be chosen, such that the particle can be separated from fluid, e.g., by filtration on with a specific pore size or by some other physical property, e.g., fluorescence.

Particles, such as microbeads, can have a diameter of less than one millimeter. For example, microbeads diameters can range from about 0.1 to about 1,000 micrometers in diameter, including as about 2-20 microns in diameter, or about 5-10 microns in diameter. Particles, such as nanobeads, can have a diameter from about 1 nanometer (nm) to about 100,000 nm in diameter, inclusive, for example, a size ranging from about 1-100 nm, or about 10-1,000 nm, or about from 200-500 nm. In certain embodiments, particles used are beads, particularly microbeads and nanobeads. Certain nucleic acid particle conjugation methods are incorporated as described in U.S. Pat. No. 7,932,037, the disclosure of which is incorporated herein by reference in its entirety.

As used herein, the term “array” refers to an arrangement of discrete features for storing, routing, amplifying and releasing oligonucleotides or complementary oligonucleotides for further reactions. The array can be planar. In an embodiment, the support or array can be addressable. Addressable supports or arrays enable the direct control of individual isolated volumes such as droplets.

As used herein, the term “immobilized” refers to oligonucleotides bound to a solid support that may be attached through their 5′ end or 3′ end. The support-bound oligonucleotides may be immobilized on the chip or bead via a nucleotide sequence (e.g., degenerate binding sequence) or linker (e.g., light-activatable linker or chemical linker). It should be appreciated that by 3′ end, it is meant the sequence downstream to the 5′ end and by 5′ end it is meant the sequence upstream to the 3′ end. For example, an oligonucleotide may be immobilized on the chip or bead via a nucleotide sequence or linker that is not involved in subsequent reactions. Certain immobilization methods are reviewed by Nimse et al., Sensors 2014, 14, 22208-22229, the disclosure of which is incorporated herein by reference in its entirety.

As used herein, the term “anchor oligonucleotide” refers to “nucleic acids,” “nucleic acid sequences,” “oligonucleotides,” “polynucleotides,” “genes” or other grammatical equivalents as used herein means at least three nucleotides, either deoxyribonucleotides or ribonucleotides, or analogs thereof, covalently linked together, and bound to a solid support that may be attached through their 5′ end or 3′ end. Anchor oligonucleotides may be immobilized on a solid support (e.g., bead, chip, particle, multi-well dish, or other substrate) via a nucleotide sequence (e.g., degenerate binding sequence) or linker (e.g., light-activatable linker or chemical linker). It should be appreciated that by 3′ end, it is meant the sequence downstream to the 5′ end and by 5′ end it is meant the sequence upstream to the 3′ end.

As used herein, the term “chemical cleavage” refers to the release of an immobilized oligonucleotide by cleaving or degrading a labile linkage susceptible to chemical cleavage or degradation, thus freeing the immobilized oligonucleotide. For example, a region of the linkage can contain a region that is chemically modified to hydrolyze or degrade in response to changes in the pH of the local environment. Certain chemically-cleavable linkers are reviewed by Leriche et al., Bioorganic and Medicinal Chemistry 20 (2012) 571-582, the disclosure of which is incorporated herein by reference in its entirety.

As used herein, the term “enzymatic cleavage” refers to the release of an immobilized oligonucleotide by cleaving or degrading a labile linkage containing a region susceptible to enzymatic degradation, thus freeing the immobilized oligonucleotide. Exemplary cleavable groups include but are not limited to peptidic sequences cleavable by proteases such as TEV protease, trypsin, thrombin, cathepsin B, cathepsin D, cathepsin K, caspase 1, and matrix metalloproteinase, as well as groups such as phosphodiester, phospholipid, ester, and β-galactose. Certain enzyme-cleavable linkers are reviewed by Leriche et al., Bioorganic and Medicinal Chemistry 20 (2012) 571-582, the disclosure of which is incorporated herein by reference in its entirety. In addition, the linkage can contain a nucleic acid sequence susceptible to cleavage by restriction enzymes. Examples of restriction enzyme cleavage sites include, but are not limited to those recognizable by common restriction enzymes such as AatI, AatIIAccI, AflII, AluI, Alw44I, ApaI, AseI, AvaI, BamHI, BanI, BanII, BanIII, BbrPI, BclI, BfrI, BglI, BglII, BsiWI, BsmI, BssHII, BsEII, BstXI, Cfr9I, Cfr10I, Cfrl3I, CspI, Csp45I, DdeI, DraI, Eco47I, Eco47III, Eco52I, Eco8lI, Eco105I, EcoRI, EcoRII, EcoRV, EcoT22I, EheI, FspI, HaeII, HaeIII, HhaI, Hin1I, HincII, HindIII, HinfI, HpaI, HpaII, KpnI, MboII, MluI, MroI, MscI, MspI, MvaI, NaeI, NarI, NciI, NcoI, NheI, NotI, NruI, NspV, PacI, PpuMI, PstI, PvuI, PvuII, RsaI, SacI, SacII, SalI, Sau3AI, Sau96I, ScaI, ScrFI, SfiI, SmaI, SpeI, SphI, SrfI, SspI, TaqI, TspEI, XbaI and XhoI.

As used herein, the term “cleavage of a light-activatable linker” refers to the release of an immobilized oligonucleotide by cleaving or degrading a labile linkage susceptible to light and/or heat from the light, such as a laser, thus freeing the immobilized oligonucleotide. For example, a region of the linkage can be degraded by heat, as a result of the application of a laser to the linkage. Other light- or photo-cleavable groups include 2-Nitrobenzyl derivatives, phenacyl ester, 8-quinolinyl benzenesulfonate, coumarin, phosphotriester, bis-arylhydrazone, and bimane bi-thiopropionic acid derivatives. Certain light-activatable linkers are reviewed by Leriche et al., Bioorganic and Medicinal Chemistry 20 (2012) 571-582, the disclosure of which is incorporated herein by reference in its entirety.

As used herein, “target” means a nucleic acid of a known nucleotide sequence (e.g., as ordered by a customer) to be identified, synthesized or assembled using one or more methods disclosed herein.

As used herein, “construction oligonucleotides” refers to oligonucleotides that can be utilized in multiplex nucleic acid assembly to generate target nucleic acids. Construction oligonucleotides can be generated by identifying a specific nucleic acid sequence and parsing the sequence into two or more fragments that can be assembled (e.g., ligated) into the desired target nucleic acid.

As used herein, “predetermined order” refers to a nucleic acid of a known nucleotide sequence (e.g., ordered by a customer or otherwise designed prior to oligonucleotide synthesis) to identify, synthesize or assemble using one or more methods disclosed herein.

As used herein, “fluorophore” or “fluorescent label” refers to a molecule that contains a functional group that can absorb radiation of a specific wavelength and emit radiation in a different, specific wavelength. A fluorophore can be physically attached to illuminate, label or identify a molecule, such as an oligonucleotide. A wide variety of fluorophores are available and applicable to the methods and devices described herein, including fluorescein, rhodamine, or cyanine based dyes and the like. For examples. a variety of dyes are commercially available and include Cy dyes available from Jackson Immuno Research (West Grove, Pa.), such as Cy2, Cy3, Cy5, and the like, or the Alexa® family of dyes available from Invitrogen/Molecular Probes (Carlsbad, Calif.), such as Alexa: 488, 500, 514, 532, 546, 555, 568, 594, 610, 633, 647, 660, 680, 700, and 750. Alternatively fluorophores such as MoFlo XDP, that are compatible with flow cytometry, are commercially available from Beckman Cutler (Danvers, Mass.).

Alternatively, labeling strategies may employ labeling moieties, such as fluorescent nanoparticles, e.g., Quantum Dots, that possess inherent fluorescent capabilities due to their semiconductor make up and size in the nanoscale regime. Such nanocrystal materials are commercially available from, e.g., Invitrogen, Inc., (Calsbad Calif.). Such compounds may be present as individual labeling groups or as interactive groups or pairs, e.g., with other inorganic nanocrystals or organicfluorophores. In some embodiments, fluorophores are incorporated as described in U.S. Pat. No. 8,133,702, which is incorporated herein by reference in its entirety.

The term “differentially labeled” or “differential label” means that the labels, e.g., fluorescent labels are different for each individual target (e.g., particle).

As used herein, “including,” “comprising,” “having,” “containing,” “involving,” and variations thereof, are meant to encompass the items listed thereafter and equivalents thereof as well as additional items. “Consisting of” shall be understood as a close-ended relating to a limited range of elements or features. “Consisting essentially of” limits the scope to the specified elements or steps but does not exclude those that do not materially affect the basic and novel characteristics of the claimed invention.

Other terms used in the fields of recombinant nucleic acid technology, synthetic biology, and molecular biology as used herein will be generally understood by one of ordinary skill in the applicable arts.

### Synthetic Oligonucleotides

Synthetic oligonucleotides can be used in multiplex nucleic acid assembly as construction oligonucleotides. To assemble a target nucleic acid, one strategy is to analyze the sequence of the target nucleic acid and parse it into two or more construction oligonucleotides that can be assembled (e.g., ligated) into the target nucleic acid.

In some embodiments, one or more construction oligonucleotides can be amplified before assembly. To facilitate amplification, one or more construction oligonucleotides and/or subconstructs may be designed to comprise one or more primer biding sites to which a primer can bind or anneal in a polymerase chain reaction. The primer biding sites can be designed to be universal (i.e., the same) to all construction oligonucleotides or a subset thereof, or two or more subconstructs. Universal primer biding sites (and corresponding universal primers) can be used to amplify all construction oligonucleotides or subconstructs having such universal primer biding sites in a polymerase chain reaction. Primer binding sites that are specific to one or more select construction oligonucleotides and/or subconstructs can also be designed, so as to allow targeted, specific amplification of the select construction oligonucleotides and/or subconstructs. In some embodiments, one or more construction oligonucleotides and/or subconstructs may contain nested or serial primer binder sites at one or both ends where one or more outer primers and inner primers can bind. In one example, the construction oligonucleotides and/or subconstructs each have binding sites for a pair of outer primers and a pair of inner primers. One or both of the pair of outer primers may be universal primers. Alternatively, one or both of the pair of outer primers may be unique primers. In some embodiments, before assembly, each of the construction oligonucleotides is individually amplified. The construction oligonucleotides can also be pooled into one or more pools for amplification. In one example, all of the construction oligonucleotides are amplified in a single pool. In certain embodiments, the amplified construction oligonucleotides are assembled via polymerase based assembly or ligation. The amplified construction oligonucleotides may be assembled hierarchically or sequentially or in a one-step reaction into the target nucleic acid.

One or more of the primer binding sites can be designed to be part of the construction oligonucleotides that are incorporated into the final target nucleic acid. In some embodiments, all or part of each primer binding site can be in the form of a flanking region outside the central portion of a construction oligonucleotide, wherein the central portion is incorporated into the final target nucleic acid and the flanking region needs be removed before assembly. To that end, one or more restriction enzyme (RE) sites can be designed to allow removal of the flanking region.

In some embodiments, the RE sites can be a type II RE sites such as type IIP or IIS and modified or hybrid sites. Type IIP enzymes recognize symmetric (or palindromic) DNA sequences 4 to 8 base pairs in length and generally cleave within that sequence. Examples: EcoRI, HindIII, BamHI, NotI, PacI, MspI, HinPlI, BstNI, NciI, SfiI, NgoMIV, EcoRI, HinfI, Cac8I, AlwNI, PshAI, BglI, XcmI, HindIII, NdeI, SacI, PvuI, EcoRV, NciI, TseI, PspGI, BglII, ApoI, AccI, BstNI, and NciI. Type IIS restriction enzymes make a single double stranded cut 0-20 bases away from the recognition site. Examples include but are not limited to BstF5I, BtsCI, BsrDI, BtsI, AlwI, BccI, BsmAI, EarI, MlyI (blunt), PleI, BmrI, BsaI, BsmBI, FauI, MnlI, SapI, BbsI, BciVI, HphI, MboII, BfuAI, BspCNI, BspMI, SfaNI, HgaI, BseRI, BbvI, EciI, FokI, BceAI, BsmFI, BtgZI, BpuEI, BsgI, MmeI, BseGI, Bse3DI, BseMI, AcIWI, Alw261, Bst6I, BstMAI, Eam1104I, Ksp632I, PpsI, SchI (blunt), BfiI, Bso31I, BspTNI, Eco31I, Esp3I, SmuI, BfuI, BpiI, BpuAI, BstV2I, AsuHPI, Acc36I, LweI, AarI, BseMII, TspDTI, TspGWI, BseXI, BstV1I, Eco57I, Eco57MI, GsuI, and BcgI. Such enzymes and information regarding their recognition and cleavage sites are available from commercial suppliers such as New England Biolabs, Inc. (Ipswich, Mass., U.S.A.).

The RE sites can be methylated such that they can be digested with a methylation-sensitive nuclease such as MspJI, SgeI and FspEI. Such nuclease shares both type IIM and type IIS properties; thus, it only recognizes the methylation-specific 4-bp sites, mCNNR (N=A or T or C or G; R=A or G), and cuts DNA outside of this recognition sequences.

Following design of the construction oligonucleotides based on the target nucleic acid, construction oligonucleotides can be synthesized or otherwise supplied by commercial vendors or any methods known in the art. Typically, oligonucleotide synthesis involves a number of chemical steps that are performed in a cycle repetitive manner throughout the synthesis with each cycle adding one nucleotide to the growing oligonucleotide chain. The chemical steps involved in a cycle are a deprotection step that liberates a functional group for further chain elongation, a coupling step that incorporates a nucleotide into the oligonucleotide to be synthesized, and other steps as required by the particular chemistry used in the oligonucleotide synthesis, such as e.g. an oxidation step required with the phosphoramidite chemistry. Optionally, a capping step that blocks those functional groups which were not elongated in the coupling step can be inserted in the cycle. The nucleotide can be added to the 5′-hydroxyl group of the terminal nucleotide, in the case in which the oligonucleotide synthesis is conducted in a 3′→5′ direction or at the 3′-hydroxyl group of the terminal nucleotide in the case in which the oligonucleotide synthesis is conducted in a 5′→3′ direction.

For clarity, the two complementary strands of a double stranded nucleic acid are referred to herein as the positive (P) and negative (N) strands. This designation is not intended to imply that the strands are sense and anti-sense strands of a coding sequence. They refer only to the two complementary strands of a nucleic acid (e.g., a target nucleic acid, an intermediate nucleic acid fragment, etc.) regardless of the sequence or function of the nucleic acid. Accordingly, in some embodiments the P strand may be a sense strand of a coding sequence, whereas in other embodiments the P strand may be an anti-sense strand of a coding sequence. It should be appreciated that the reference to complementary nucleic acids or complementary nucleic acid regions herein refers to nucleic acids or regions thereof that have sequences which are reverse complements of each other so that they can hybridize in an antiparallel fashion typical of natural DNA.

In some aspects of the disclosure, the oligonucleotides synthesized or otherwise prepared according to the methods described herein can be used as building blocks for the assembly of a target polynucleotide of interest.

Oligonucleotides may be synthesized on solid support using methods known in the art. In some embodiments, pluralities of different single-stranded oligonucleotides are immobilized at different features of a solid support. In some embodiments, the support-bound oligonucleotides may be attached through their 5′ end or their 3′ end. In some embodiments, the support-bound oligonucleotides may be immobilized on the support via a nucleotide sequence (e.g. degenerate binding sequence), linker (e.g. photocleavable linker or chemical linker). It should be appreciated that by 3′ end, it is meant the sequence downstream to the 5′ end and by 5′ end it is meant the sequence upstream to the 3′ end. For example, an oligonucleotide may be immobilized on the support via a nucleotide sequence or linker that is not involved in subsequent reactions.

Certain embodiments of the disclosure may make use of a solid support comprised of an inert substrate and a porous reaction layer. The porous reaction layer can provide a chemical functionality for the immobilization of pre-synthesized oligonucleotides or for the synthesis of oligonucleotides. In some embodiments, the surface of the array can be treated or coated with a material comprising suitable reactive group for the immobilization or covalent attachment of nucleic acids. Any material, known in the art, having suitable reactive groups for the immobilization or in situ synthesis of oligonucleotides can be used.

In some embodiments, the porous reaction layer can be treated so as to comprise hydroxyl reactive groups. For example, the porous reaction layer can comprise sucrose.

According to some aspects of the disclosure, oligonucleotides terminated with a 3′ phosphoryl group oligonucleotides can be synthesized a 3′→5′ direction on a solid support having a chemical phosphorylation reagent attached to the solid support. In some embodiments, the phosphorylation reagent can be coupled to the porous layer before synthesis of the oligonucleotides. In an exemplary embodiment, the phosphorylation reagent can be coupled to the sucrose. For example, the phosphorylation reagent can be 2-[2-(4,4′-Dimethoxytrityloxy)ethylsulfonyl]ethyl-(2-cyanoethyl)-(N,N-diisopropyl)-phosphoramidite. In some embodiments, the 3′ phosphorylated oligonucleotide can be released from the solid support and undergo subsequent modifications according to the methods described herein. In some embodiments, the 3′ phosphorylated oligonucleotide can be released from the solid support using ammonium hydroxide.

In some embodiments, synthetic oligonucleotides for the assembly may be designed (e.g. sequence, size, and number). Synthetic oligonucleotides can be generated using standard DNA synthesis chemistry (e.g., phosphoramidite method). Synthetic oligonucleotides may be synthesized on a solid support, such as for example a microarray, using any appropriate technique as described in more detail herein. Oligonucleotides can be eluted from the microarray prior to be subjected to amplification or can be amplified on the microarray. It should be appreciated that different oligonucleotides may be designed to have different lengths.

In some embodiments, oligonucleotides are synthesized (e.g., on an array format) as described in U.S. Pat. No. 7,563,600, U.S. patent application Ser. No. 13/592,827, and PCT/US2013/047370 published as WO 2014/004393, which are hereby incorporated by reference in their entireties. For example, single-stranded oligonucleotides are synthesized in situ on a common support wherein each oligonucleotide is synthesized on a separate or discrete feature (or spot) on the substrate. In some embodiments, single-stranded oligonucleotides are bound to the surface of the support or feature. As used herein, the term “array” refers to an arrangement of discrete features for storing, routing, amplifying and releasing oligonucleotides or complementary oligonucleotides for further reactions. The array can be planar. In an embodiment, the support or array is addressable: the support includes two or more discrete addressable features at a particular predetermined location (i.e., an “address”) on the support. Therefore, each oligonucleotide molecule of the array is localized to a known and defined location on the support. The sequence of each oligonucleotide can be determined from its position on the support. Moreover, addressable supports or arrays enable the direct control of individual isolated volumes such as droplets. The size of the defined feature can be chosen to allow formation of a microvolume droplet on the feature, each droplet being kept separate from each other. As described herein, features are typically, but need not be, separated by interfeature spaces to ensure that droplets between two adjacent features do not merge. Interfeatures will typically not carry any oligonucleotide on their surface and will correspond to inert space. In some embodiments, features and interfeatures may differ in their hydrophilicity or hydrophobicity properties.

An oligonucleotide may be a single-stranded nucleic acid. However, in some embodiments a double-stranded oligonucleotide may be used as described herein. In certain embodiments, an oligonucleotide may be chemically synthesized as described herein. In some embodiments, synthetic oligonucleotide may be amplified before use. The resulting product may be double stranded.

One or more modified bases (e.g., a nucleotide analog) can be incorporated. Examples of modifications include, but are not limited to, one or more of the following: methylated bases such as cytosine and guanine; universal bases such as nitro indoles, dP and dK, inosine, uracil; halogenated bases such as BrdU; fluorescent labeled bases; non-radioactive labels such as biotin (as a derivative of dT) and digoxigenin (DIG); 2,4-Dinitrophenyl (DNP); radioactive nucleotides; post-coupling modification such as dR-NH2 (deoxyribose-NEb); Acridine (6-chloro-2-methoxiacridine); and spacer phosphoramides which are used during synthesis to add a spacer “arm” into the sequence, such as C3, C8 (octanediol), C9, C12, HEG (hexaethlene glycol) and C18.

In various embodiments, the synthetic single-stranded or double-stranded oligonucleotides can be non-naturally occurring, e.g., being unmethylated or modified in a way (e.g., chemically or biochemically modified in vitro) such that they become hemi-methylated (only one strand is methylated) or semi-methylated (only a portion of the normal methylation sites are methylated on one or both strands) or hypomethylated (more than the normal methylation sites are methylated on one or both strands), or have non-naturally occurring methylation patterns (some of the normal methylation sites are methylated on one or both strands and/or normally unmethylated sites are methylated). In contrast, naturally-occurring DNA typically contains epigenetic modifications such as methylation at, e.g., the C-5 position of the cytosine ring of DNA by DNA methyltransferases (DNMTs) in vivo. DNA methylation is reviewed by Jin et al., Genes & Cancer 2011 June; 2(6): 607-617, which is incorporated herein by reference in its entirety.

### Multiplex Nucleic Acid Assembly

Multiplex nucleic acid assembly can be used to prepare one or more target nucleic acids, wherein for each target, multiple construction oligonucleotides can be brought into contact with one another according to a predesigned order. The construction oligonucleotides can be single stranded wherein by design, they alternate between positive and negative strands and one partially anneals with the next such that together, they can form a double-stranded product. The construction oligonucleotides can also be double stranded and be designed to have compatible cohesive ends that at least partially anneal with one another to align the construction oligonucleotides in a predesigned order to form a double-stranded product. The double-stranded product can be gap free and produce the target nucleic acid upon ligation. The double-stranded product may contain gaps that can be filled in by a polymerase.

In some embodiments, assembly can be done in a parallel fashion where multiple target nucleic acids are prepared simultaneously. For example, 2-100,000, 5-10,000, 10-1000, or 100-500, or any other number of targets can be produced in parallel.

Assembly can be carried out using hierarchical, sequential and/or one-step assembly. By way of example only, hierarchical assembly of oligonucleotides A, B, C and D (each a construction oligonucleotide) into a A+B+C+D target may include assembling A+B and C+D first (each a subconstruct or subassembly), then A+B+C+D. Sequential assembly may include assembling A+B (a primary subconstruct or subassembly), then A+B+C (a secondary subconstruct or subassembly), and finally A+B+C+D (target). One-step assembly combines A, B, C and D in one reaction to produce A+B+C+D. It should be noted that different strategies can be mixed where a portion of the construction oligonucleotides are assembled using one strategy while another portion a different strategy.

The construction oligonucleotides can be chemically synthesized, e.g., on a solid support as described above. In some embodiments, the construction oligonucleotides can be synthesized in sufficient amount so as to enable direct subassembly or total assembly without the need to amplify one or more of the construction oligonucleotides. In certain embodiments, the construction oligonucleotides, after chemical synthesis, may be first subject to subassembly into subconstructs, which can be amplified (e.g., in a polymerase based reaction) and then subject to further assembly into secondary subconstructs or the final target. In some embodiments, one or more construction oligonucleotides can be amplified before assembly. To that end, the construction oligonucleotides can be designed to have one or more universal or specific primer binding sites as disclosed herein.

Assembly can be performed on a solid support, optionally assisted by microfluidic devices such as those disclosed in PCT Publication Nos. WO2011/066185 and WO2011/056872, the disclosure of each of which is incorporated herein by reference in its entirety.

### Bead-Based Assembly and Singulation

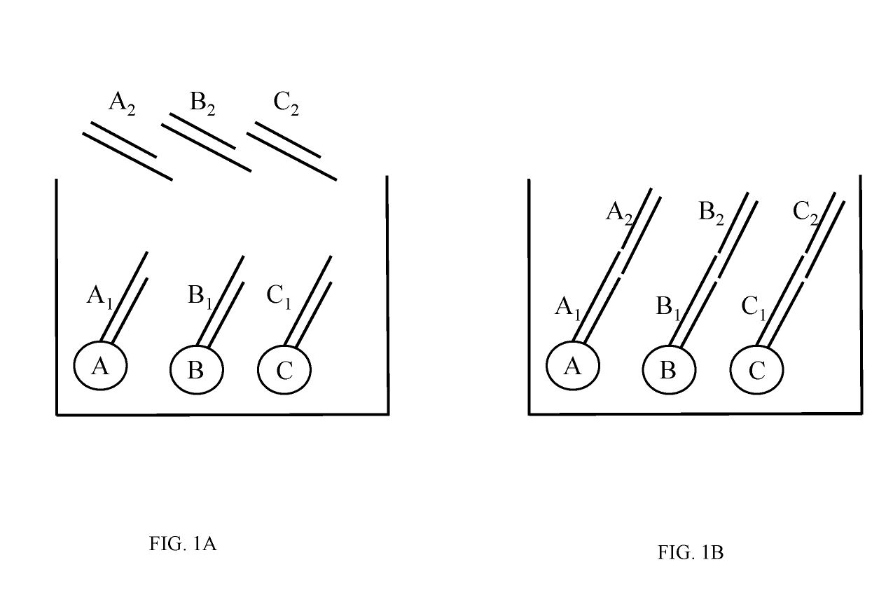

One strategy for multiplex nucleic acid assembly is to attach terminal construction oligonucleotides to beads or other individualized solid supports, while assembling the other free, non-attached construction oligonucleotides with, sequentially or in a one-pot reaction, the terminal construction oligonucleotides attached to beads, thereby producing one or more target polynucleotides. Alternatively, anchor oligonucleotides that are not part of the target polynucleotides can be attached to beads, which can serve as linker between the construction oligonucleotides and the beads. For example, referring to FIG. 1A, beads A, B, C are designed to have anchor or terminal construction oligonucleotides A₁, B₁, C₁ attached thereon. A₁, B₁, C₁ are illustrated as double-stranded but it should be understood that they can be single-stranded as well.

Referring to FIGS. 1A and 1B, beads A, B, C . . . can have (terminal) construction oligonucleotides A₁, B₁, C₁ . . . attached thereto, respectively. Further, a second set of construction oligonucleotides A₂, B₂, C₂ . . . can be assembled with A₁, B₁, C₁ . . . , respectively. Additional sets of construction oligonucleotides can be added until the final set, Aₙ, Bₙ, Cₙ . . . (n is an integer >=2 such as 2, 3, 4, 5, 6, 7, 8, 9, 10, 20, 30, etc.) are assembled to form target nucleic acids X_{A} (having sequence A₁A₂ . . . Aₙ), X_{B} (having sequence B₁B₂ . . . Bₙ), X_{C} (having sequence C₁C₂ . . . Cₙ), etc. Alternatively, all n sets of construction oligonucleotides (A₁, B₁, C₁ . . . ), (A₁, B₁, C₁ . . . ) . . . (Aₙ, Bₙ, Cₙ . . . ) can be mixed together in a single-pot assembly reaction, to form target nucleic acids X_{A}, X_{B}, X_{C} . . . .

In some embodiments, it may be desirable to separate the beads A, B, C . . . from one another, before, during, and/or after assembly, to isolate the construction oligonucleotide, the subconstruct and/or the final assembly product. One method is limiting dilution where the mixture of beads is sufficiently diluted such that when an aliquot is placed into a well, that aliquot does not contain more than one bead. From there, the nucleic acid attached to the singular bead can be isolated and identified (e.g., by sequencing). However, when the starting mixture contains a multiplicity of beads A, a multiplicity of beads B, a multiplicity of beads C . . . , limiting dilution generates many duplicate aliquots having the same bead. Since the duplicate aliquots must all be analyzed to identify the nucleic acids attached thereto, this method can be time consuming and costly.

To save time and cost, another method is to differentially label beads A, B, C . . . in a way such that they can be separated from one another. An exemplary differential label is fluorophore. A wide variety of fluorophores are commercially available and can be used to label different beads so that they can be sorted using flow cytometry (e.g., MoFlo™ XDP from Beckman Cutler). In certain embodiments, a 2-fluorochore system can be designed to label the beads. Specifically, two fluorophores can be selected and attached to different beads at different ratios, such that when excited by laser, different beads have different emission spectra due to the fluorophores associated thereto. By way of example, fluorophores F1 and F2 can be present on bead A at, in relative amount, 10% and 90%, respectively; on bead B at 15% and 85%, respectively; on bead C at 20% and 80%, respectively . . . . The difference in emission spectra can be pre-designed to be significant enough for gating the different beads in flow cytometry. The gated beads can be placed onto multi-well plates for further analysis such as sequencing the nucleic acids on the beads.

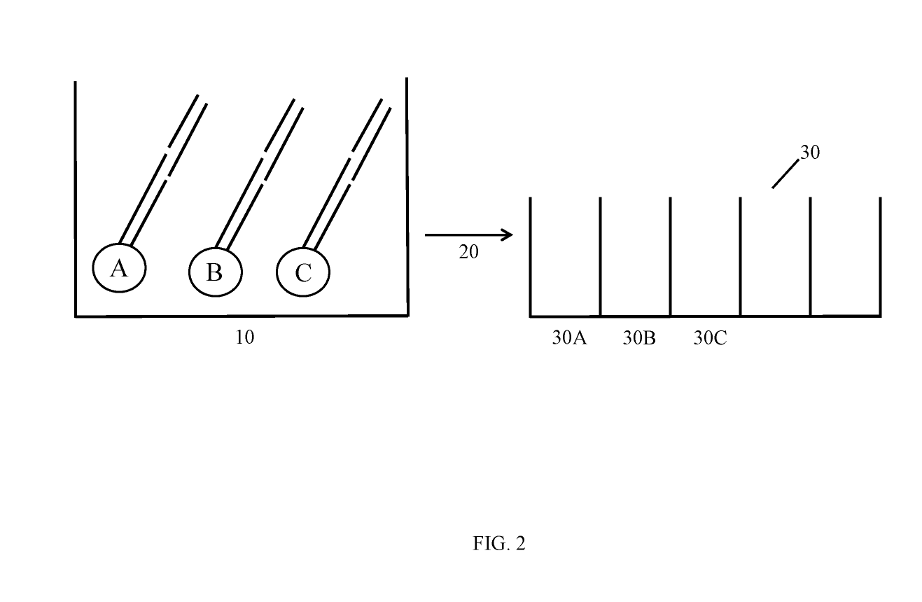

For example, FIG. 2 illustrates an exemplary method of assembled oligonucleotide singulation and sorting. Beads A, B, C can contain differential labels as disclosed herein. After assembly in reaction vessel 10, the fully assembled, differentially labeled, target oligonucleotides can then be gated according to their unique emission spectra by flow cytometry 20. The gated beads can be placed in multi-well plates 30, into individual wells 30A, 30B, 30C . . . .

It should be noted that selective singulation can be performed after complete assembly, and/or during assembly where one or more subconstructs can be singulated for further manipulation such as amplification, sequencing and/or further assembly. In addition, construction oligonucleotides, prior to assembly, can also be selectively singulated for amplification, sequencing and/or assembly.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments. Also, the phraseology and terminology used herein is for the purpose of description and should not be regarded as limiting.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for the use of the ordinal term) to distinguish the claim elements.

### INCORPORATION BY REFERENCE

All publications, patents and sequence database entries mentioned herein are hereby incorporated by reference in their entireties as if each individual publication or patent was specifically and individually indicated to be incorporated by reference.

1. A device for multiplex nucleic acid assembly, comprising:
a) a plurality of differentially labeled particles comprising a first particle, a second particle and a third particle, having a first anchor oligonucleotide, a second anchor oligonucleotide and a third anchor oligonucleotide immobilized thereon, respectively;
b) a plurality of first construction oligonucleotides designed to at least partially anneal to one another in a first predetermined order, and to at least partially anneal to the first anchor oligonucleotide at a first terminal oligonucleotide, thereby forming a first nucleic acid on the first particle;
c) a plurality of second construction oligonucleotides designed to at least partially anneal to one another in a second predetermined order, and to at least partially anneal to the second anchor oligonucleotide at a second terminal oligonucleotide, thereby forming a second nucleic acid on the second particle; and
d) a plurality of third construction oligonucleotides designed to at least partially anneal to one another in a third predetermined order, and to at least partially anneal to the third anchor oligonucleotide at a third terminal oligonucleotide, thereby forming a third nucleic acid on the third particle.

2. The device of claim 1, wherein the first, second and third particles each have a different fluorophore attached thereto.

3. The device of claim 1, wherein the first, second and third particles each have a different mixture of two fluorophores attached thereto.

4. The device of any one of claims 1-3, wherein the plurality of differentially labeled particles are provided in a single reaction volume.

5. The device of claim 4, wherein the first, second and third construction oligonucleotides are designed to have uniquely complementary sequences to one another.

6. The device of claim 1, wherein each particle is provided in a separate reaction volume.

7. The device of claim 6, wherein each separate reaction volume is provided in a different well.

8. A method for multiplex nucleic acid assembly, comprising:
a) providing a plurality of differentially labeled particles comprising a first particle, a second particle and a third particle, having a first anchor oligonucleotide, a second anchor oligonucleotide and a third anchor oligonucleotide immobilized thereon, respectively;
b) providing a plurality of first construction oligonucleotides designed to at least partially anneal to one another in a first predetermined order, and to at least partially anneal to the first anchor oligonucleotide at a first terminal oligonucleotide, thereby forming a first nucleic acid on the first particle;
c) providing a plurality of second construction oligonucleotides designed to at least partially anneal to one another in a second predetermined order, and to at least partially anneal to the second anchor oligonucleotide at a second terminal oligonucleotide, thereby forming a second nucleic acid on the second particle; and
d) providing a plurality of third construction oligonucleotides designed to at least partially anneal to one another in a third predetermined order, and to at least partially anneal to the third anchor oligonucleotide at a third terminal oligonucleotide, thereby forming a third nucleic acid on the third particle.

9. The method of claim 8, wherein the first, second and third particles each have a different fluorophore attached thereto.

10. The method of claim 8, wherein the first, second and third particles each have a different mixture of two fluorophores attached thereto.

11. The method of any one of claims 8-10, wherein the plurality of differentially labeled particles are provided in a single reaction volume.

12. The method of claim 11, wherein the first, second and third construction oligonucleotides are designed to have uniquely complementary sequences to one another.

13. The method of claim 8, wherein each particle is provided in a separate reaction volume.

14. The method of claim 13, wherein each separate reaction volume is provided in a different well.
